(19) United States
(12) Patent Application Publication
(10) Pub. No.: US 2015/0075256 A1
(43) Pub. Date: Mar. 19, 2015

(54) MULTIPLE GAS SENSOR

(71) Applicant: Crowcon Detection Instruments Limited, Abingdon, Oxfordshire (GB)

(72) Inventors: Paul Basham, Abingdon (GB); Roger Hutton, Abingdon (GB)

(21) Appl. No.: 14/382,358

(22) PCT Filed: Feb. 28, 2013
(86) PCT No.: PCT/GB2013/050489

§ 371 Date: Sep. 2, 2014

(30) Foreign Application Priority Data
Mar. 2, 2012 (GB) 1203699.2

(51) Int. Cl. G01N 33/00

(52) U.S. Cl. CPC G01N 33/0004
USPC 73/31.01

(57) ABSTRACT

A gas detector for determining the contributions of two or more target gases in an atmosphere to be sampled, the gas detector comprising: a gas sensor for sampling the gas, the gas sensor having a sensitivity to a target gas which is dependent on the temperature of the gas sensor; a temperature regulating device configured to change the temperature of the gas sensor; calculating means in communication with the gas sensor; wherein the gas detector is configured to: sample the atmosphere with the gas sensor at a plurality of temperatures, wherein the temperature regulating device changes the temperature of the sensor; and determine using the calculating means the contributions of the two or more target gases based on the measurements at the plurality of gas sensor temperatures and the sensitivity of the gas sensor at the plurality of gas sensor different temperatures.

### FIELD OF INVENTION

The present invention relates to a method and apparatus to determine the contribution of two or more target gases in an atmosphere.

### BACKGROUND TO THE INVENTION

It is known in commercially available systems to determine the level of a target gas or analyte using an electrochemical sensor (or equivalent). Such sensors are typically configured to determine with a reasonable level of accuracy the presence of an individual target gas. However in practice the sensors are typically placed in an environment which there may be several different gases, many of which will need to be monitored in order to ensure safety. For example, in a metal foundry, there may be found high concentrations of hydrogen and carbon monoxide, and the levels of both gases would need to be monitored on a regular basis in order to ensure safety.

In modern instrumentation the output of a gas sensor is typically a current. Variations in the current output is indicative of the amount of a target gas present in the sampled atmosphere. At a given temperature the total response is the sum of the contributions of the target analyte and interfering gases.

It is found that for many sensors there is a “cross sensitivity” between two gases. In such cases it may be difficult to separate the contribution from each gas in the total current response of the sensor. For example, it is known that in typically commercial available carbon monoxide sensors that such sensors are sensitive to both carbon monoxide and hydrogen. In environments such as metal foundries where both types of gas could be present, it is difficult for a single sensor to accurately determine the relative contributions of the carbon monoxide and hydrogen gases due to the cross sensitivity of the sensors. Accordingly, this results in a measure of uncertainty due to the mixture of the gases.

It is often necessary to have two or more different types of gas sensor, each type of sensor configured to measure a specific target gas. The requirements of individual sensors to determine the presence of a specific gas results in increased cost and associated maintenance costs.

Furthermore, the cross sensitivity of a sensor to two or more gases may result in less accurate readings returned by a gas detector. As such gas detectors are placed in potentially dangerous environments, ensuring the accuracy of the readings from such a gas detector is of importance.

In order at least mitigate at least one of the above mentioned problems regarding the prior art, there is provided a gas detector for determining the contributions of two or more target gases in an atmosphere to be sampled, the gas detector comprising: a gas sensor for sampling the gas, the gas sensor having a sensitivity to a target gas which is dependent on the temperature of the gas sensor; a temperature regulating device configured to change the temperature of the gas sensor; wherein the gas detector is configured to: sample the atmosphere with the gas sensor at a plurality of temperatures, using the temperature regulating device to change the temperature of the sensor.

There is also provided a method of determining the contributions of two or more target gases in an atmosphere to be sampled, the method comprising the steps of: sampling the gas with a gas sensor, the gas sensor at a first temperature and the gas sensor having a sensitivity to a target gas which is dependent on the temperature of the gas sensor; changing the temperature of the gas sensor with a heating device; and sampling the gas with the gas sensor at a second different temperature. Preferably, wherein the range of the plurality of gas sensor temperatures is between −30° C. to 50° C.

Preferably the initial temperature of the sensor is the temperature of the gas to which the sensor is exposed (i.e. the initial temperature of the gas sensor is approximately the same as the gas to which it is measuring). Therefore the sensor is configured to function at around ambient temperature and does not require significant temperature change in order to measure the relative contributions of the gases. As only a relatively small amount of heating, or cooling, is required the sensor does not represent an ignition or combustion risk if placed in a hazardous environment.

Other aspects of the invention will become apparent from the appended claim set.

### BRIEF DESCRIPTION OF THE FIGURES

Embodiments of the invention are now described, by way of example only, with reference to the accompanying drawing in which:

FIG. 1 is a schematic diagram of a gas detector according to an embodiment of the invention;

FIGS. 2a and 2b are plots of the sensitivity to gas sensors to CO and H₂ over different temperatures; and

FIG. 3 is a flow chart of the process of determining the contribution of two or more target gases with a gas sensor.

### DETAILED DESCRIPTION OF AN EMBODIMENT

There is described above a process of thermal control of a gas sensor which improves the selectivity and sensitivity of the sensor with respect to a plurality of gases. In particular, the present invention allows for the determination of a plurality of target gases with a single sensor, and also compensates for any cross sensitivity in a given sensor between two types of gases which would typically result in uncertainty in the measurements regarding the mixture of gases.

The response by a particular sensor to a particular gas may vary according to the temperature of the sensor. In particular it is found that sensors typically show much greater sensitivity to a given target gas at a particular temperature range (the temperature range being dependent on the gas being measured and the type of sensor). In the situation where a cross sensitivity may result from the presence of two or more gases in an atmosphere to be tested, for example Carbon Monoxide (CO) and Hydrogen (H₂), it has been beneficially realised that the relative contribution of each gas may be determined by sampling the atmosphere to be tested at a number of temperatures. If the response of the sensor to a gas at a given temperature is known then by measuring the atmosphere over a range of temperatures then the contribution for each gas can be determined. In an example the electrochemical sensor is a potentiostatically or galvanostically controlled electrochemical sensor.

Such gas detectors are designed to be either portable or fixed gas detectors which are typically placed in hazardous environments. Such environments may contain explosive gases, and/or gases which when inhaled are fatal to humans. Therefore, the detector must be able to work safely in such an environment. As described below the sensor is configured to function at temperatures which near ambient, and accordingly do not represent a combustion hazard.

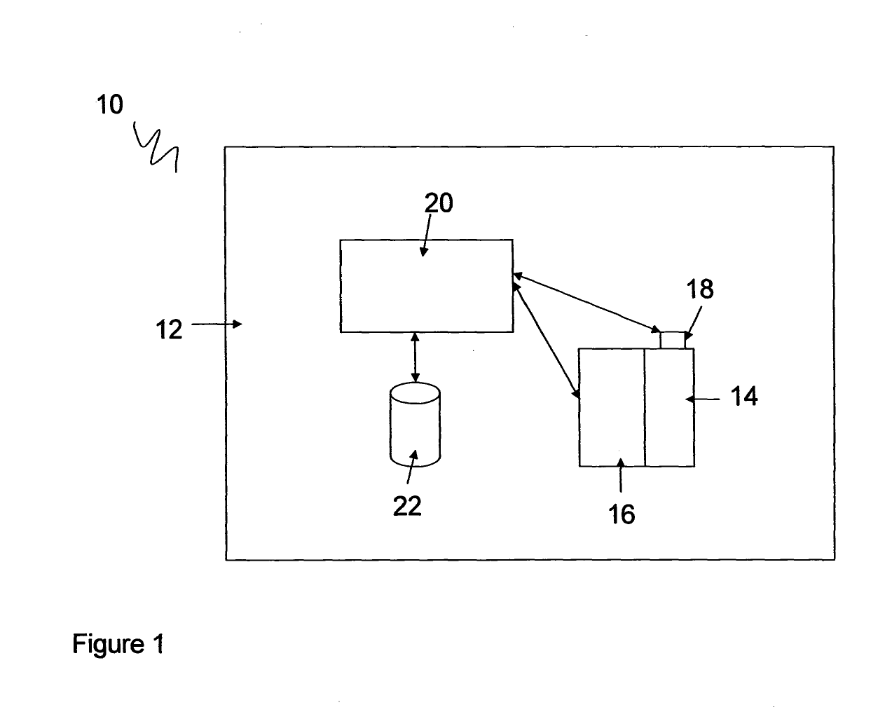

FIG. 1 is a schematic of a gas detector in an example of the invention.

There is shown: the gas detector 10; having a housing 12; inside the housing is stored a sensor 14; a heat regulating device; a temperature measuring device 18; a processor 20 which is in communication with the heat regulating device 16 and temperature measuring device 18 and a form of memory 22 preferably at least part of which is non-volatile memory.

The detector 10 is a variant of known commercially available gas detectors. In the present example, the gas sensor is an electrochemical gas sensor stored in the housing unit 12. The sensor 14 has a small surface area and is constructed from a material having a high thermal conductivity such as a ceramic. e.g. alumina. For example, the sensor may be a sensor from a manufacturer such as Solidsense. The material preferably is selected from a material with a thermal conductivity of at least 20 Wm⁻¹K⁻¹.

In an example, such detectors are gas sensors, preferably portable, which are designed to be placed in environments such as oil refineries, mines, metal foundry plants etc. Such environments are typically corrosive environments and the housing 12 is a thermoplastic. The housing 12 therefore is designed to withstand impacts and/or the corrosive chemicals to which the detector 10 is typically exposed.

As described above, the temperature of the sensor 14 is varied in order to measure the gas at a number of different sensor temperatures. This can be achieved by using a heat regulating device 16 (such as a thermostat coupled to the sensor 14) and the temperature measuring device 18 (which is also coupled to the sensor 14). The working range of the temperature sensor is between −30° C. to 50° C. and more preferably the sensor operates between 10° C. to 50° C., and preferably the initial temperature is at the ambient temperature of the gas it is measuring (i.e. the sensor does not require heating or cooling before initial use).

Conventional detectors have sensors typically made from plastic, electrolyte and metallic electrodes, or other materials which have a high thermal capacity and accordingly require a large amount of energy to change the temperature. Due to the high thermal capacity, the rate of change in temperature of such sensors is typically low. As gas sensors typically sample an atmosphere several times a minute, (reasons for which are described below) the embodiments with a slow change in temperature are not as advantageous.

Beneficially the change in the temperature sensor is within a period which is shorter than the exposure of the gas to the sensor. Preferably, this is within a period of insignificant change in the level of gas present at the sensor, of the order of 10 seconds. The sensor 14 used can have a small surface area and a high thermal conductivity in order to ensure that the change of temperature can occur over a short period of time. Preferably, the sensor incorporates high thermal conductivity ceramics which are found to be able to change the temperature of the sensor over a period of seconds.

In order to change the temperature of the sensor 14, the heat regulating device 16 is coupled to the sensor 14. The heat regulating device 16 is configured to heat and cool the sensor 14. In a preferred example, the cooling occurs using Peltier cooling. Alternatively, a sterling engine or other known cooling methods can be used. The heating of the sensor 14 occurs via resistive heating, though other heating methods can be used. The temperature measuring device 18 is coupled to the sensor 14 and is configured to determine the temperature of the sensor in a known manner.

The heat regulating device 16 and temperature measuring device 18 are in communication with the processor 20. The processor 20 monitors the temperature of the sensor 14 via the temperature measuring device 18 and is configured to regulate the temperature of the sensor 14 by the heat regulating device 16. The processor 20 can increase or decrease the temperature of the sensor 14 and measure the atmosphere to be sampled across a number of sensor 14 temperatures. The processor 20 is stored within the housing 12 of the detector. Alternatively, the processor is not placed within the housing 12 and the detector 10 comprises a bi-directional communication device (not shown) which is in communication with a central computer (not shown) having a processor. The central computer receiving information regarding the temperature of the sensor and the sensor output. In such an embodiment, the central computer is able to calculate the relative contributions of two or more target gases using the information sent via the detector 10.

The processor 20 or any other form of calculating device or means, is configured to calculate the relative contribution of two or more gases across a number of different sensor temperatures. This includes, but is not limited to, the use of look up tables hard coded into hardware or stored on non-volatile memory, microprocessors, algorithmic logic units and integrated circuits and any other form of processing unit.

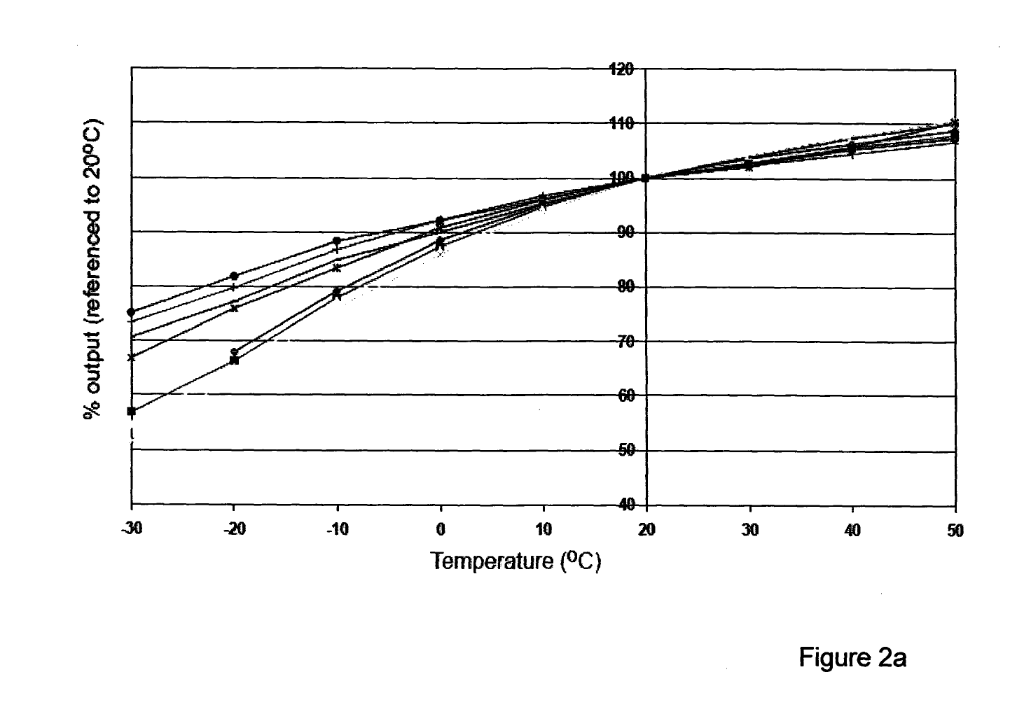

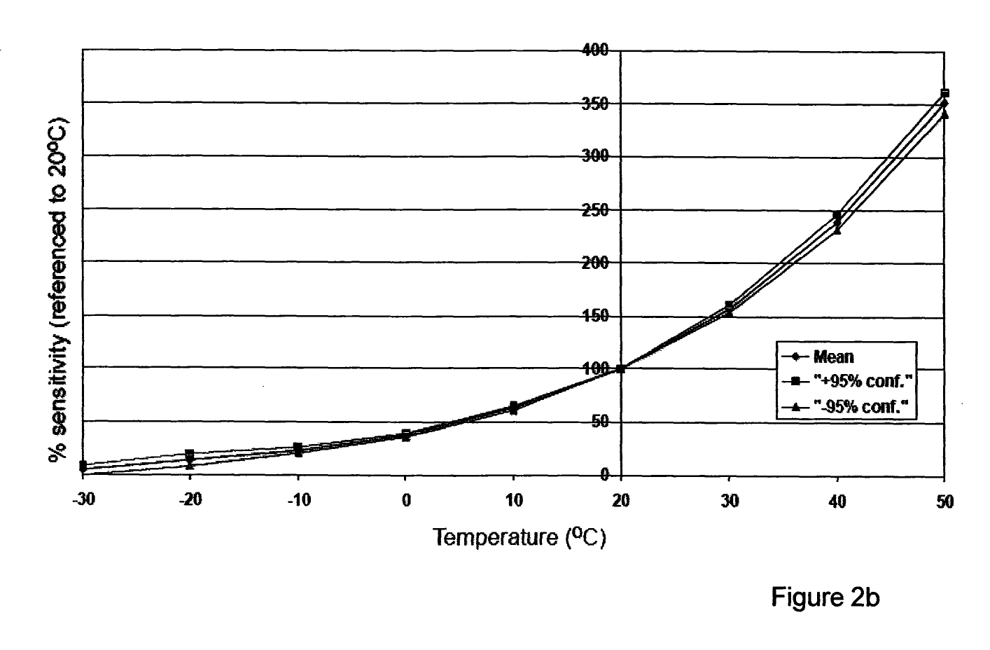

FIGS. 2a and 2b show the difference in sensitivity to two targets gases of a batch of commercially available electrochemical carbon monoxide sensors due to cross sensitivity over a large temperature range.

FIG. 2a shows the CO response versus temperature and FIG. 2b shows the H₂ response for the same sensors over the same temperature range.

As can be clearly seen in both Figures the output of the sensor for either gas is temperature dependent, and shows a different dependency based on gas type.

In FIG. 2a the response of the sensors between 20° C. and 50° C. displays an increase in sensitivity to CO of 10%. As can be seen in FIG. 2b, the sensitivity of the same electrochemical sensors to H₂ increases greatly over the same temperature range. The sensors show an increased sensitivity to H₂ with temperature, increasing by 250% between the same temperature range of 20 to 50° C. Accordingly, it is clear from the plots that the carbon monoxide sensors display a much greater sense of sensitivity to hydrogen at higher temperatures (e.g. greater than 35° C.) than they do to carbon monoxide.

By measuring the presence of gas using a relatively high temperature of the sensor (e.g. greater than 35° C.), the sensor is much more sensitive to H₂ than to CO so it can be assumed that the readings from H₂ are the dominate source of current in the sensor response and that the CO response makes a minimal contribution to the sensor. Therefore a sensor reading at a high temperature may be indicative of the level of H₂ present.

In contrast to known laboratory based MOS systems, the invention therefore functions at temperatures around ambient temperature (e.g. between −10° C. to 50° C., and more preferably between +10° C. or 20° C. to 50° C.). Preferably the initial temperature of the sensor is the temperature of the gas to which the sensor is exposed (i.e. the initial temperature of the gas sensor is approximately the same as the gas to which it is measuring). Therefore the sensors used do not require significant heating in order to measure the gas. Furthermore, as the detector may be placed in environments which contain explosive gases it is desirable to ensure that all components are kept at temperatures lower than the ignition temperature of the gases in order to improve the safety of the device.

Similarly, referring to FIG. 2b when the temperature of the carbon monoxide sensor is reduced to below 5° C. the sensor is much more sensitive to CO at these temperatures than to H₂ and a reading at these temperatures is indicative of the level of CO as the H₂ will make a minimal contribution.

Again, in contrast to known MOS sensors, or pellistor technologies, the described apparatus allows for the ability to measure the presence of multiple gases by varying the temperature of the sensor at temperatures around ambient temperature (e.g. between −30° C. to 50° C., and more preferably between +10° C. or 20° C. to 50° C.). The use of the Sterling engine or Peltier device allows for such a temperature range to be maintained using the sensor having the high thermal conductivity.

By measuring the atmosphere to be tested across a range of sensor temperatures around ambient or room temperature, the relative contributions of each of the two gases may be determined, as the sensors' response to the gases at different temperatures is known beforehand.

It is possible to determine the relative contribution of a number of gases, by using simultaneous equations or look up tables.

The invention can determine the relative contributions of two or more target gases by varying the temperature of a gas sensor. In an example, the gas detector comprises a known electrochemical gas sensor, though in other embodiments other forms of sensor are used.

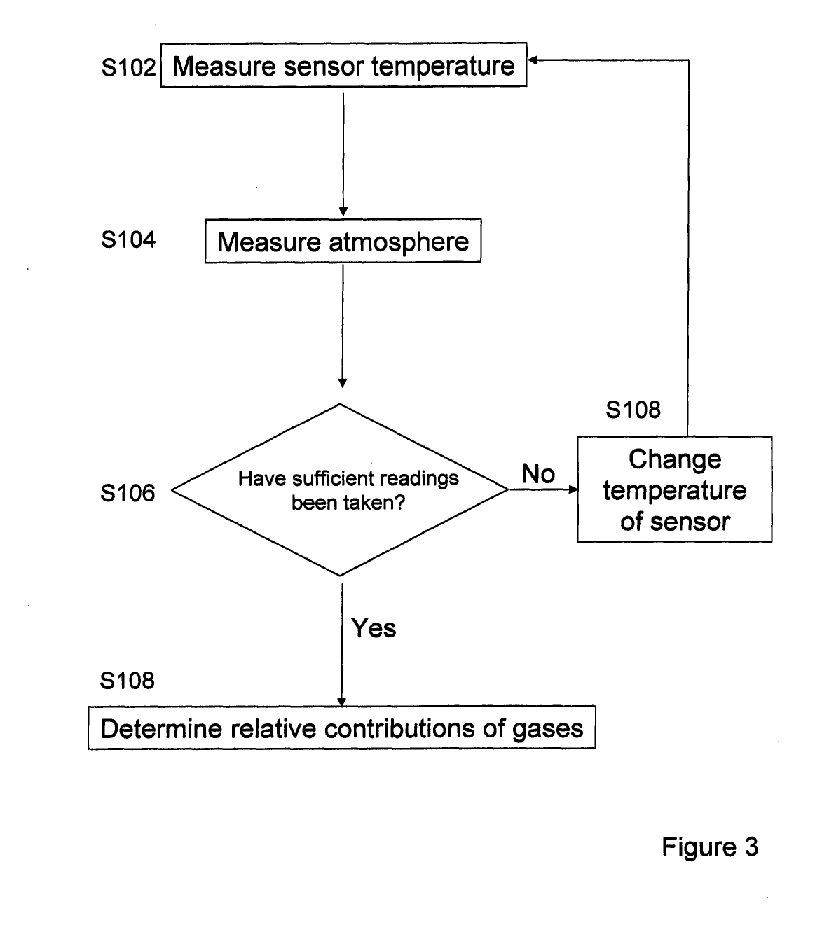

FIG. 3 is a flowchart of a process for measuring the relative contributions of two or more target gases in an atmosphere to be sampled.

There is shown the step of S102 measuring the gas sensor temperature, at step S104 measuring the gas at the given sensor temperature, at step S106 determining if sufficient readings have been made to be able to accurate calculate the relative contributions of two or more target gases, at step S108 changing the temperature of the sensor, and step S110 determining the relative contributions of each gas.

As described above, such commercially available sensors have a variable response which is dependent on the sensor temperature. At step S102 the temperature of the gas sensor is measured. The temperature at which the gas is measured initially is preferably at the ambient temperature of the sensor, which is dependent on the environment in which the sensor is placed (typically between −10° C. to +50° C., more typically around room temperature). Once measured, the temperature is stored in a form of writable memory 22 associated with the gas detector. The memory may be stored within the gas detector, or may be part of a computer system in communication with the gas sensor.

At step S104, the atmosphere to be measured is measured using the gas sensor. In known electrochemical sensors, the response of the sensor is determined by the current emitted by the sensor. As described with reference to FIG. 1, the response of the sensor (i.e. the outputted current) due to the presence of a particular gas is dependent on the temperature of the sensor. Therefore at step S104 the temperature of the sensor 14 (as measured at step S102) and the output current are stored in the writeable memory 22 associated with the gas sensor 14. Preferably at step S104 the initial temperature of the sensor is at approximately the ambient temperature of the gas, so the sensor does not require significant heating or cooling).

As the relative contributions of two or more target gases are to be determined using this method, there are at least two unknown variables (i.e. the relative contribution of each of the target gases to be measured) which need to be determined. In an example, simultaneous equations are used to determine the values of the unknown variables. In order to solve the simultaneous equations, multiple readings are taken at different sensor temperatures. At step S106, the processor determines whether there is sufficient data in order to determine the relative contributions of each of the target gases at the range of temperatures. If it is not possible to solve the simultaneous equations with a sufficient degree of accuracy, then the process moves to step S108, and if there are sufficient data points in order to solve the simultaneous equations, the process moves to step S110.

At step S108, the temperature of the sensor is changed using a temperature regulating device 16. The temperature regulating device 16 is coupled to the sensor 14 and is configured to change the temperature of the sensor 14 either via known cooling or heating methods. The cooling can occur via Peltier cooling, a sterling engine or other methods may also be used. Heating is achieved by way of resistive heating or other known methods e.g. reversing the voltage across a Peltier cooler. By using these heating and cooling methods the temperature of the sensors is kept at temperatures near ambient (i.e. across the range as shown in FIGS. 2a and 2b, and preferably between 10 to 50° C.). Therefore, the sensor is kept at close to ambient temperature ensuring the sensor does not provide an ignition risk.

Once the temperature of the gas sensor has been changed at step S108, the process returns to step S102 (measuring sensor temperature) and reiteratively repeats between steps S102 and steps S108 until there is sufficient data in order to determine the relative contributions (as determined at step S106) has been met. At such time, it is possible to determine the relative contributions of each gas (i.e. solve the simultaneous equations to determine the values of each of the unknown variables).

In an example, the change to the temperature of the sensor at step S108 is of the order of 5 K and occurs over a timescale of 5 seconds. The total change in temperature range is preferably is no more than 20K preferably less than 10K. Accordingly as the initial temperature of the sensor is at ambient temperature the sensor works at around room temperature.

Preferably the change in temperature of the sensor and subsequent measurements of the sensor occur in timescales of the order of seconds. Preferably, the change in temperature of the sensor is less than the time that gas is exposed to the sensor. This ensures that the same sample of gas is measured through all temperature ranges.

It is found that such heating and cooling may be performed efficiently using gas sensors which have high thermal conductivity, low thermal capacity and a low surface area. This allows for a rapid change in the temperature of the sensor of the order of seconds.

At step S110 the relative contributions of each gas are determined. The levels of gas are determined, in an example, using simultaneous equations. The following example is given by way of example only.

The temperature change method could equally be applied to a variety of sensor types e.g. electrochemical sensors, pellistors. There is prior art for the application to micro-hotplates and MOS metal oxide semiconductor sensors (see paper attached to mail).

For the case of a CO sensor in which the H₂ sensitivity is strongly temperature dependent (as shown FIG. 2) the sensitivity to a particular gas at a particular temperature (T) is given by

s=gas(T).

In order to efficiently calculate the relative contributions of the gases the response of the sensor is determined experimentally beforehand. In such experiments the sensor is placed in an environment which contains a known concentration of the target gases and the sensor's response is measured for the known gas concentration over a number of different sensor temperatures. In an example the sensor's response is experimentally determined at 20° C. and 50° C., thus the following information is known. The actual figures have been inserted by way of example, and the skilled person would realise that the actual figures will vary according to the type of sensor used.

s_{CO}(20)=86

s_{CO}(50)=96

s_{H2}(20)=4

s_{H2}(50)=14

The composition of H₂ and CO in a gas may be expressed as x_{H2}+y_{CO}. The response of the sensor will be the total measured current z, and is expressed as z=y·s_{CO}+x·s_{H2}. i.e. the total contribution of the output of the sensor will include contributions of both the CO and H₂ at a given temperature. As the sensitivity of the sensor at different temperatures is known it is therefore possible to use this information to determine the relative contributions of each gas.

At 20° C. the total current z₂₀ is given by s_{CO}(20)·y+sH₂(20)·x

At 50° C. the total current z₅₀ is given by s_{CO}(50)·y+sH₂(50)·x

By substituting in the known predetermined values, it is possible to solve the equations for x and y, allowing the gas composition to be determined. In the above example:

z₅₀=96y+14x

z₂₀=86y+4x

Rearranging for x

x=(z₂₀−86y)/4

Therefore

z₅₀=96y+14(z₂₀−86y)/4

4z₅₀=344y+14z₂₀−86y

Hence

y=(4z50−14z20)/258

In other embodiments other correlation methods are used to determine the components of the gas. In an example, the signal is modulated (e.g. sinusoidally) and the resultant signal is correlated. Thus the relative contributions of each gas, and therefore their amounts, are determined.

Whilst the above example has been given with respect to two unknown variables the same principles can be applied to measure a number (e.g. more than two) of target gases. Furthermore, by sampling the atmosphere at a number of different temperatures the contributions of each gas can be calculated multiple times. This allows for the determined answers to be compared with each other in order to check the accuracy of the reading. Optionally, known mathematical techniques to identify and remove outliers are employed to improve the accuracy of the readings.

Whilst the above example has been described with reference to the use of simultaneous equations in order to determine the relative contributions of each of the gases, it is also possible to determine the contributions of the relevant gases by determining the response of a sensor to a target gas in laboratory conditions and look up tables. In such embodiments the response of the detectors are calculated across a range of temperatures for a number of known different atmospheric compositions. These values are stored in the memory associated with the detector. Therefore in use the detector will measure the atmosphere to be sampled at a number of different temperatures and the response of the sensor at each temperature measured. The values of the response at a range of temperatures are compared to those previously calculated for a number of different atmospheric compositions and the most likely atmospheric composition is determined from the predetermined values.

In use the detector 10 continuously samples the atmosphere, preferably at a rate of 10 times a minute. Thus allowing for any changes in the composition of the atmosphere to be readily identified. Therefore, in a preferred embodiment the sensor 14 is made from a material with a low thermal capacity and a high thermal conductive allowing the sensor to be heated and cooled over relatively short timescales, of the order of seconds. This allows the above method and apparatus to be continuously used and for a prolonged period of time.

In an example the determined contributions are indicated to a user via a visual indicator such as an LCD display (not shown). If the levels of gas are determined to be outside of a predetermine safe range, then a sensory alarm (e.g. flashing light, audible alarm etc) indicates that a dangerous level of a target gas has been detected.

The above described apparatus and method allow for the sampling of multiple gases such as O₂, CH₄, NOₓ, NH₃, CO, H₂, and CO₂, to be accurately sampled simultaneously with a single sensor.

Most commercially available electrochemical sensors can be categorised into one of three groups. Each group showing for a sensor significant cross sensitivity that exists for the sensor between the gases in the same group. The groups may be classified as follows:

Group 1 O₃, Cl₂, ClO₂, HCl, HF, F₂, Br₂, HBr, NO₂;

Group 2 H₂, CO, H₂S, ETO; and

Group 3 SO₂, HCN, PH₃ ethylene, alcohols.

Therefore, the invention provides a method and apparatus to allow the concentrations of one or more target gases to be determined using the same sensor. Furthermore, the techniques described above allow for the more accurate determination of the concentration of the gases as cross-sensitivity between two or more gases is accounted for and corrected.

Advantageously the sensor works at ambient temperature and the temperature range across which the gas is sampled is low (preferably of the order of ˜20K, more preferably less than 10K). Accordingly, the temperature range at which the sensor operates does not pose an ignition threat to the gas which it is intended to sample.

In an example, such detectors are portable gas sensors which are designed to be placed in relatively inhospitable environments such as oil refineries, mines, metal foundry plants etc.

In further examples, pellistors and other catalytic gas sensors that are commonly used to detect flammable gas of a variety of types may also use this method with a change in temperature sensor range. Ammonia alcohols methane, propane, ethylene and jet fuel may be distinguished this way.

1.-22. (canceled)

23. A gas detector for determining the contributions of two or more target gases in an atmosphere to be sampled, the gas detector comprising:
a gas sensor for sampling the gas, the gas sensor having a sensitivity to a target gas which is dependent on the temperature of the gas sensor;
a temperature regulating device configured to change the temperature of the gas sensor;
a processor;
wherein the gas detector is configured to change the temperature of the sensor, thereby changing the sensitivity of the sensor to the target gas, and sample the atmosphere with the gas sensor at a plurality of temperatures, using the temperature regulating device to change the temperature of the sensor, and wherein the detector further comprises a processor in communication with the gas sensor; and
wherein the detector is further configured to determine, using the processor, the contributions of the two or more target gases based on the measurements at the plurality of gas sensor temperatures and the sensitivity of the gas sensor at the plurality of gas sensor different temperatures, wherein the range of the plurality of gas sensor temperatures is between −30° C. to 50° C.

24. The gas detector of claim 23 wherein the initial temperature of the gas sensor is at the ambient temperature of the gas to which the gas sensor is exposed.

25. The gas detector of claim 23 wherein the range of gas sensor temperatures is between +10° C. to 50° C.

26. The gas detector of claim 23 wherein the change of temperature occurs over a timescale shorter than the period of time the gas is exposed to the sensor.

27. The gas detector of claim 26 wherein the period of time is of the order of 10 seconds or less.

28. The gas detector of claim 23 wherein the gas sensor has a thermal conductivity greater than 20 Wm⁻¹K⁻¹.

29. The gas detector of claim 23 wherein the sensor comprises a high thermal conductivity ceramic.

30. The gas detector of claim 23 wherein the temperature regulating devices is a heat pump or Peltier device or Stirling engine.

31. The gas detector of claim 23 wherein the gas detector further comprises a memory and the pre-determined sensitivity of the gas sensor to one or more gases at different temperatures for one or more known atmospheric composition(s) is stored on the memory.

32. The gas detector of claim 31 wherein the determination of the relative contributions is calculated by comparing the measured response at the plurality of different temperatures for the unknown atmospheric composition with the pre-determined response of the gas sensor for the known atmospheric compositions stored in the memory and identifying the most likely atmospheric compositions based on the comparison.

33. The gas detector of claim 31 wherein the determination of the relative contributions is calculated by measuring the response of the sensor at a plurality of temperatures for an unknown atmospheric composition being tested and comparing with the values of the pre-determined response for a known atmospheric composition and solving using simultaneous equations the composition of the atmosphere being tested.

34. The gas detector of claim 23 wherein the detector is configured to determine the contribution of one or more of the group selected from:
O₃, Cl₂, ClO₂, HCl, HF, F₂, Br₂, HBr, NO₂; or
a group selected from H₂, CO, H₂S, ETO; or
a group selected from 3 SO₂, HCN, PH₃ ethylene, alcohols.

35. A method of determining the contributions of two or more target gases in an atmosphere to be sampled, the method comprising the steps of:
sampling the gas with a gas sensor, the gas sensor having a sensitivity to a target gas which is dependent on the temperature of the gas sensor, at a first temperature and;
changing the temperature of the gas sensor with a heating device, thereby changing the sensitivity of the sensor to the target gas;
sampling the gas with the gas sensor at a second different temperature; and
determining, using a processor, the contributions of the two or more target gases based on the measurements at the plurality of gas sensor temperatures and the sensitivity of the gas sensor at the plurality of gas sensor different temperatures, wherein the range of the plurality of gas sensor temperatures is between −30° C. to 50° C.

36. The method of claim 35 wherein the initial temperature of the gas sensor is at the ambient temperature of the gas to which the gas sensor is exposed.

37. The method of claim 35 wherein the range of gas sensor temperatures is between +10° C. to 50° C., preferably between 20° C. and 50° C.

38. The method of claim 35 wherein the method determines the contribution of one or more of the group selected from:
O₃, Cl₂, ClO₂, HCl, HF, F₂, Br₂, HBr, NO₂; or
a group selected from H₂, CO, H₂S, ETO; or
a group selected from 3 SO₂, HCN, PH₃ ethylene, alcohols.

39. The method of claim 35 wherein the sensor is a pellistors or other type of catalytic gas sensor, and the method determines the contribution of one or more flammable gases.

40. The method of claim 39 wherein the flammable gases are selected from the group containing: ammonia alcohols methane, propane, ethylene and jet fuel.
